(19) United States
(12) Patent Application Publication
(10) Pub. No.: US 2026/0209820 A1
(43) Pub. Date: Jul. 23, 2026

(54) APPARATUSES, SYSTEMS, AND METHODS FOR TESTING CATHETERS

(71) Applicant: Innovotech, Inc., Edmonton (CA)

(72) Inventor: Brenton Stanford Buziak, Edmonton (CA)

(73) Assignee: Innovotech, Inc., Edmonton (CA)

(21) Appl. No.: 19/448,911

(22) Filed: Jan. 14, 2026

Related U.S. Application Data
(60) Provisional application No. 63/746,578, filed on Jan. 17, 2025.

(51) Int. Cl. C12Q 1/06; A61M 25/00

(52) U.S. Cl. CPC C12Q 1/06; A61M 25/0017

(57) ABSTRACT

Apparatuses, systems, and methods for testing a catheter include an outer tubing for receiving the catheter; and a stand including a reticulating holder for receiving the outer tubing and catheter therein, and clamping and releasing means for (i) securing the holder, outer tubing, and catheter into a first “U” shaped position for conditioning and/or challenging internal and external surfaces of the catheter simultaneously, and (ii) releasing the holder, outer tubing, and catheter into a second “L” shaped position for draining fluid. Sealable end caps isolate the catheter shaft within the outer tubing, and facilitate draining fluid from one end of the outer tubing through a spout without spillage. Sealable air vents are elevated above the fluid in the outer tubing to prevent contamination. A removable cover maintains sterility of catheter components external from the outer tubing.

### CROSS REFERENCE TO RELATED APPLICATIONS

This application claiming the benefit of priority to U.S. Patent Application Ser. No. 63/746,578, filed Jan. 17, 2025, the contents of which are hereby incorporated by reference in their entirety.

### FIELD OF THE INVENTION

The present invention relates generally to apparatuses, systems, and methods for testing catheters. In particular, the invention relates to apparatuses, systems, and methods for in vitro testing of the internal and external surfaces of catheters simultaneously to evaluate reduction in microbial colonization.

### BACKGROUND OF THE INVENTION

Catheters are flexible medical instruments intended for the withdrawal and introduction of fluids relative to body cavities, ducts, and vessels. Catheter instrumentation may have specific applications in various medical procedures. However, infection is a potentially-life threatening complication of catheterization. Microbial colonization of a catheter increases daily upon placement in a patient, particularly for catheters that cross, and are maintained at, the skin-air interface. There is a higher risk of catheter colonization and associated complications in long-term catheterized patients, with contamination frequently occurring on the exterior of the catheter from the insertion site, or the interior of the catheter due to poor technique during fluid withdrawal or introduction. Microbial colonization also occurs with urinary catheters including Foley catheters, endotracheal tubes, and drainage ports for drainage tubes. Preclinical evaluation of novel or existing catheters designed to prevent such infections needs to assess microbial colonization accurately in vitro before clinical trials. Further, in vitro simulation of microbial colonization may help in selecting suitable treatments for minimizing or preventing microbial colonization in vivo.

Attempts in the prior art for testing catheters typically require a continuous flow of media through a catheter testing apparatus or system, thereby increasing the cost for testing due to the volume of media required, and rendering the testing unfeasible at the scale and within the timeframe required for completion of regulatory submissions. Specific media such as, for example, human serum or plasma, may be difficult to procure in the large volumes required for a continuous flow catheter testing apparatus or system. Accordingly, there remains a need for improved apparatuses, systems, and methods for testing catheters which may overcome many of the shortcomings of existing technologies.

### SUMMARY OF THE INVENTION

The present invention relates generally to apparatuses, systems, and methods for testing catheters. In particular, the invention relates to apparatuses, systems, and methods for in vitro testing of the internal and external surfaces of catheters simultaneously to evaluate reduction in microbial colonization.

Notably, the apparatuses, systems, and methods are conducted using minimal set volumes of fluid (e.g., media, inoculum, etc.), thereby obviating the need for a continuous flow of fluid and the associated expense, time, and waste. The apparatus has been specially designed to operate with minimal set volumes of fluid, to be sealed in such a way as to prevent or reduce the risk of contamination and spillage, and to be incubated with or without agitation for predetermined time periods before draining and replacing the fluid.

In particular, the apparatus can be easily filled, incubated, drained, and re-filled, using minimal set volumes of fluid. An outer tubing is configured for receiving a catheter to be tested. A stand includes a reticulating holder for receiving the outer tubing and catheter therein, and clamping and releasing means for (i) securing the holder, outer tubing, and catheter in a first “U” shaped position for in vitro testing (i.e., conditioning and challenging) of the internal and external surfaces of the catheter simultaneously to evaluate reduction in microbial colonization, and (ii) releasing the holder, outer tubing, and catheter in a second “L” shaped position for draining fluid.

The apparatus is sealable to prevent or reduce the risk of contamination and spillage of fluid. Sealable end caps on the outer tubing isolate the catheter shaft within the outer tubing, and facilitate draining the fluid from one end of the outer tubing through a spout without spillage. Sealable air vents are elevatable above the fluid in the outer tubing to avoid contamination by the fluid. A removable cover maintains sterility of other catheter components (i.e., those typically kept outside of the patient's body) external from the outer tubing.

Broadly, in one aspect, an apparatus for testing a catheter comprises:
an outer tubing configured for receiving the catheter; and
a stand comprising a reticulating holder configured for receiving the outer tubing and the catheter therein, and clamping and releasing means for (i) securing the holder, the outer tubing, and the catheter into a first “U” shaped position for conditioning and/or challenging internal and external surfaces of the catheter simultaneously, and (ii) releasing the holder, the outer tubing, and the catheter into a second “L”-shaped position for draining fluid.

In some embodiments, the reticulating holder comprises a plurality of links coupled by a plurality of movable joints and defining a channel for receiving the outer tubing and the catheter therein.

In some embodiments, the outer tubing and the catheter are formed of flexible materials. In some embodiments, the catheter comprises a catheter hub, an elongated catheter member extending distally from the catheter hub, one or more extension lines, and one or more luer hubs. In some embodiments, the outer tubing comprises a first tubing end, a second tubing end, and an elongated tubing body extending between the first and second tubing ends and defining a tubing bore for receiving and accommodating the catheter therethrough.

In some embodiments, the outer tubing further comprises first and second tubing caps removably attachable at the first and second tubing ends for sealing the catheter in the tubing bore.

In some embodiments, the first tubing cap comprises an upwardly extending arm for removably attaching a first sealable air vent extending upwardly above the fluid in the outer tubing.

In some embodiments, the apparatus further comprises a first end cap removably attachable to the first tubing cap, the first end cap defining a port for allowing the catheter member to pass therethrough, and isolating the catheter hub, the extension lines, and the luer hubs externally from the outer tubing.

In some embodiments, the apparatus further comprises a cover for encasing the first tubing cap, the first end cap, the catheter hub, the extension lines, and the luer hubs.

In some embodiments, the second tubing end comprises a spout including a cylindrically-shaped housing defining a central outlet, a shoulder, external threads, and a conduit extending therethrough for allowing the drainage of the fluid from the outer tubing. In some embodiments, the central outlet projects upwardly from the shoulder and has a diameter narrower than the diameter of the shoulder. In some embodiments, the external threads of the spout mate with corresponding internal threads of a second end cap.

In some embodiments, the second tubing cap comprises a cylindrically-shaped housing defining a cavity for receiving the spout, internal threads for engaging the external threads of the spout, an internal seat for resting against the shoulder of the spout, a central aperture allowing passage of air from the outer tubing through a second sealable air vent, and a gasket for sealing the spout against the second tubing cap.

In some embodiments, the second end cap is removably attachable to the second tubing cap and comprises upwardly projecting walls defining internal threads for engaging external threads of the second sealable air vent extending upwardly above the fluid within the outer tubing.

In some embodiments, the stand further comprises a base including a top portion and a bottom portion, wherein the top portion comprises an upper surface for supporting a pair of opposed clips for detachably retaining the reticulating holder. In some embodiments, the stand further comprises a frame for mounting first and second clamp arms including first and second clamping means for clamping the reticulating holder, the outer tubing, and the catheter.

In another aspect, a method for testing a catheter comprises:
providing the above catheter and apparatus;
optionally, conditioning lumens of the catheter shaft and an extraluminal portion of the catheter shaft within the outer tubing with one or more conditioning fluids;
challenging with a medium comprising a microbial inoculum by filling the lumens of the catheter shaft and the outer tubing with the medium to contact the internal and external surfaces of the catheter shaft simultaneously;
incubating the apparatus for a predetermined time sufficient for microbial colonization on the internal and external surfaces of the catheter shaft;
collecting the medium from the lumens of the catheter shaft and the outer tubing, the medium comprising planktonic microorganisms collected in an initial recovery step, or microorganisms dislodged from the internal and external surfaces of the catheter shaft collected in a subsequent recovery step; and
quantifying microbial colonization from the collected medium.

In some embodiments, the catheter is selected from a hemodialysis catheter, a central venous catheter, a peripherally inserted central catheter, a peripheral intravenous catheter, an endotracheal tube, or a Foley catheter. In some embodiments, the hemodialysis catheter comprises an acute hemodialysis catheter.

In some embodiments, conditioning comprises pre-treating and incubating the lumens of the catheter shaft and the extraluminal portion of the catheter shaft within the outer tubing with the one or more conditioning fluids for a predetermined time. It is envisioned that multiple conditioning fluid changes may be made, and the rate at which they are changed may be designed to mimic clinically relevant conditions. In some embodiments, the one or more conditioning fluids are selected from saline, infusate, human plasma, human serum, artificial urine, or artificial saliva. In some embodiments, the method further comprises draining the one or more conditioning fluids from the lumens of the catheter shaft and the outer tubing before challenging with the medium comprising the microbial inoculum.

Additional aspects and advantages of the present invention will be apparent in view of the description which follows. It should be understood, however, that the detailed description and the specific examples, while indicating preferred embodiments of the invention, are given by way of illustration only, since various changes and modifications within the scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a perspective front view of a first embodiment of an apparatus for testing a catheter, showing all components when assembled.

FIG. 2 is a perspective front view of the apparatus of FIG. 1 which is partially assembled, showing the outer tubing and catheter held in the “L” shaped position, within the holder of the stand, for draining fluid.

FIG. 3 is a perspective front view of two apparatuses of FIG. 1, when stacked by using two stands.

FIG. 4 is a perspective front view showing assembly of components including the catheter, cover, and outer tubing.

FIG. 5 is a schematic diagram showing a cross-sectional side view of outer tubing and the catheter contained within when assembled.

FIG. 6 is a perspective front view of the apparatus of FIG. 1 partially assembled, showing the outer tubing and catheter held in the “U” shaped position within the holder of the stand.

FIG. 7A is a perspective front view of a portion of the apparatus of FIG. 2, showing a spout at the second tubing end of the outer tubing. FIG. 7B is a perspective top view of the spout of FIG. 7A.

FIG. 8 is a perspective bottom view of a second tubing cap configured to cover the spout of FIG. 7B.

FIG. 9 is a perspective front view of a portion of the apparatus of FIG. 2, showing a second air vent positioned behind the cover.

FIG. 10 is a perspective front view of a second embodiment of an apparatus for testing a catheter, showing all components when assembled.

FIG. 11 is a perspective side view of the apparatus of FIG. 10, with the front portion of the cover removed to show the first end cap at the first tubing end of the outer tubing.

FIG. 12 is a perspective side view of the apparatus of FIG. 10, with the second tubing cap removed to show a spout at the second tubing end of the outer tubing.

FIG. 13 is a perspective front view of the apparatus of FIG. 10, showing the outer tubing and catheter held in the “L” shaped position, within the holder of the stand, for draining fluid.

FIG. 14 is a perspective front view of the apparatus of FIG. 10, showing the outer tubing and catheter held in the “L” shaped position, within the holder of the stand, for draining fluid. The front portion of the cover and the second tubing cap are removed to show the first end cap at the first tubing end of the outer tubing, and the spout at the second tubing end of the outer tubing.

FIG. 15A is a perspective front view of a stand. FIG. 15B is a perspective front view of the apparatus of FIG. 10 within the stand of FIG. 15A. FIG. 15C is a perspective front view of two stands of FIG. 15A, when stacked. FIG. 15D is a perspective front view of two apparatuses of FIG. 10, when stacked using the stands of FIG. 15C.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a,” “an,” and “the” include plural referents unless the context clearly dictates otherwise. The term “horizontal” means the orientation of a plane or line that is substantially parallel to the plane of the horizon. The term “vertical” means the orientation of a plane or line that is substantially at a right angle to the horizontal plane.

The present invention relates generally to apparatuses, systems, and methods for testing catheters. In particular, the invention relates to apparatuses, systems, and methods for in vitro testing of the internal and external surfaces of catheters simultaneously to evaluate reduction in microbial colonization.

Broadly, in one aspect, an apparatus for testing a catheter comprises:
an outer tubing configured for receiving the catheter; and
a stand comprising a reticulating holder configured for receiving the outer tubing and the catheter therein, and clamping and releasing means for (i) securing the holder, the outer tubing, and the catheter into a first “U” shaped position for conditioning and/or challenging internal and external surfaces of the catheter simultaneously, and (ii) releasing the holder, the outer tubing, and the catheter into a second “L” shaped position for draining fluid.

In another aspect, a method for testing a catheter comprises:
providing the above catheter and the apparatus;
optionally, conditioning lumens of the catheter shaft and an extraluminal portion of the catheter shaft within the outer tubing with one or more conditioning fluids;
challenging with a medium comprising a microbial inoculum by filling the lumens of the catheter shaft and the outer tubing with the medium to contact the internal and external surfaces of the catheter shaft simultaneously;
incubating the apparatus for a predetermined time sufficient for microbial colonization on the internal and external surfaces of the catheter shaft;
collecting the medium from the lumens of the catheter shaft and the outer tubing, the medium comprising planktonic microorganisms collected in an initial recovery step, or microorganisms dislodged from the internal and external surfaces of the catheter shaft collected in a subsequent recovery step; and
quantifying microbial colonization from the collected medium.

As used herein, the term “catheter” broadly refers to any medical catheter for the administration of fluids (withdrawal or introduction) relative to the body of a subject. In some embodiments, the catheter is selected from a hemodialysis catheter, a central venous catheter, a peripherally inserted central catheter, a peripheral intravenous catheter, an endotracheal tube, or a Foley catheter. In some embodiments, the catheter comprises an acute hemodialysis catheter (“AHDC”) or a multi-lumen AHDC. An AHDC provides venous access for hemodialysis, apheresis, rapid fluid administration, intravenous therapy, blood sampling, pressure injection of contrast media, central venous pressure monitoring, hemofiltration, and hemoperfusion. However, it is envisioned that the present disclosure may be employed with a range of catheter applications including surgical, diagnostic and related treatments of diseases and body ailments of a subject. It is further envisioned that the principles relating to the catheter disclosed include employment with various catheter related procedures, such as, for example, hemodialysis, cardiac, abdominal, urinary, intestinal, endotracheal, and in chronic and acute applications. Moreover, the catheter can be used for administration or removal of fluids such as, for example, medication, saline, bodily fluids, blood and urine. It is further envisioned that the invention may be similarly used with drains and various drain related procedures.

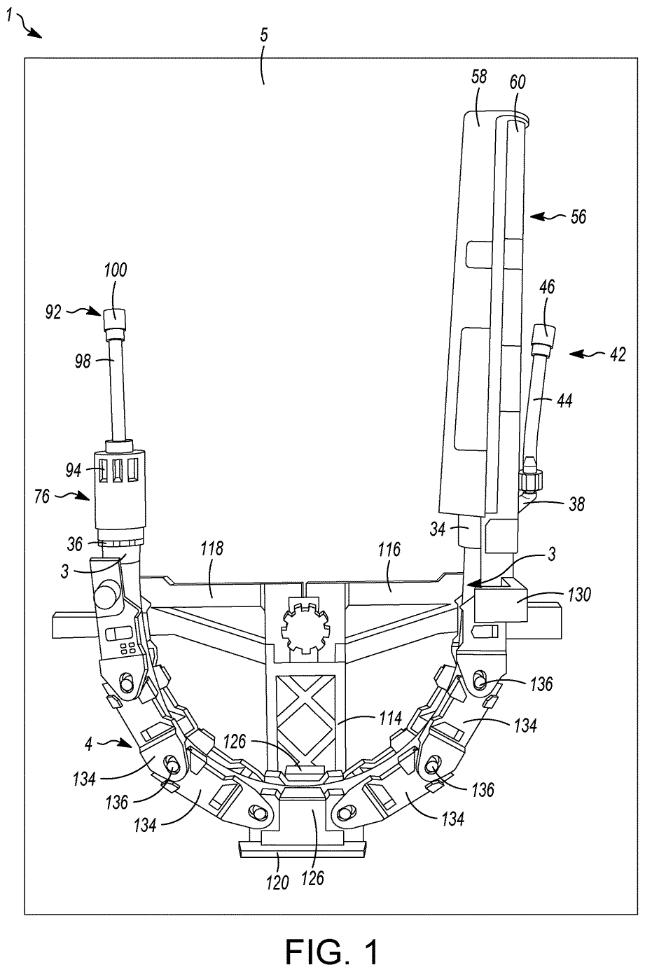

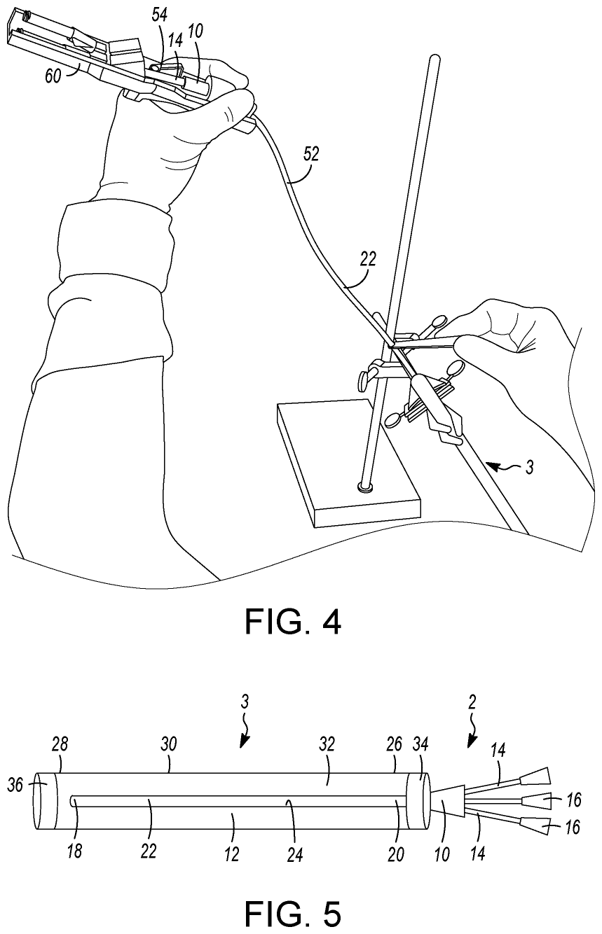

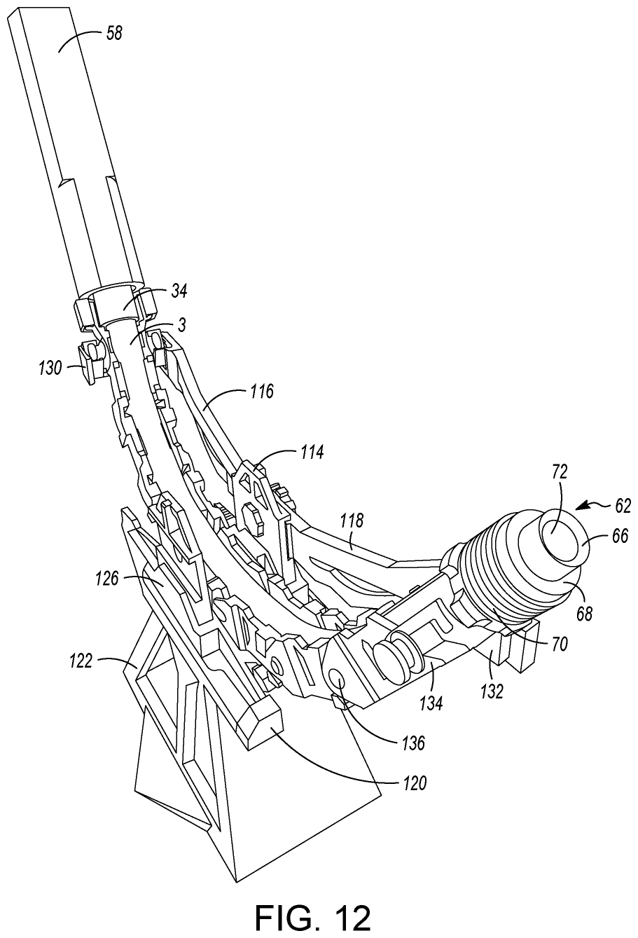

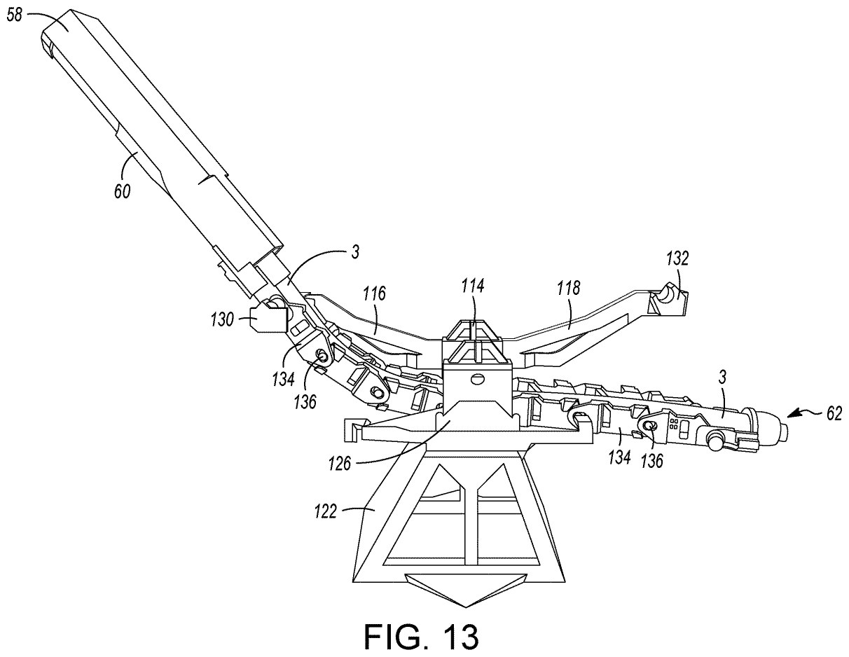

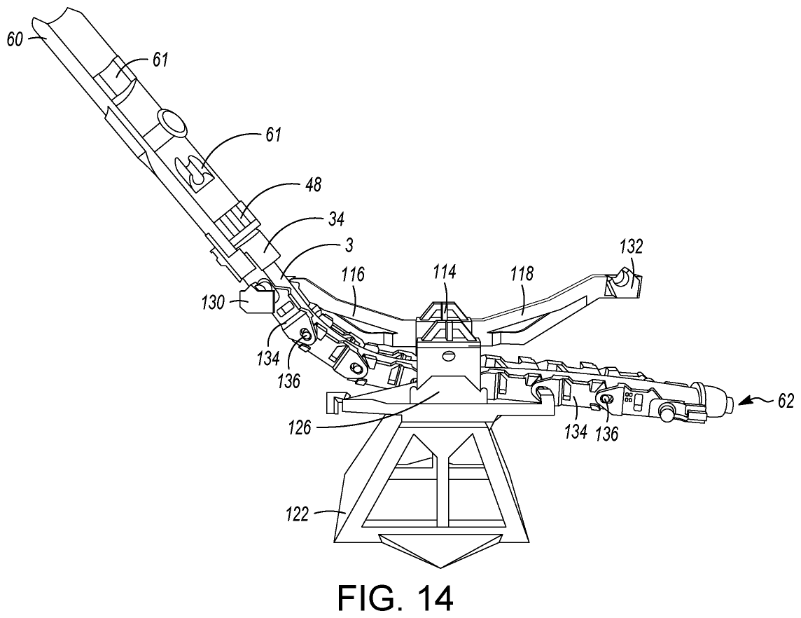

The invention will now be described having reference to the accompanying Figures. The apparatus (1) of the present invention is shown assembled in FIGS. 1-3 and 10. The apparatus (1) is shown partially assembled in FIGS. 4, 6, and 11-14. FIGS. 5, 7A-9, and 15A-D show specific components of the apparatus (1) in greater detail.

In some embodiments, the catheter (2) includes several assembled components including a catheter hub (10) and an elongated catheter member (12) extending distally from the catheter hub (10) (FIG. 5). In some embodiments, the catheter hub (10) is frustoconically-shaped. The catheter hub (10) is connected to one or more extension lines (14) which serve as venous and arterial lines leading, for example, to a conventional hemodialysis machine. Each extension line (14) is connected to a corresponding luer hub (16). In some embodiments, each luer hub (16) is frustoconically-shaped. The luer hub (16) serves as a connection for the transfer of fluids from tubing (not shown) of the hemodialysis machine or other equipment (not shown) to the extension lines (14) and the catheter member (12).

The catheter member (12) comprises a distal end (18), a proximal end (20), and a catheter shaft (22) extending between the distal and proximal ends (18, 20). The distal end (18) is configured to be introduced through a desired entry site of the subject's body. In some embodiments, the distal end (18) comprises a pliable tip. The catheter shaft (22) defines one or more lumens (24), each of which extends throughout the length of the catheter shaft (22) for allowing the passage of fluid(s). Suitable fluids include, but are not limited to, medium, saline, infusate, human plasma, human serum, artificial urine, artificial saliva, inoculum, and the like.

For clarity, a single lumen (24) is shown in FIG. 5. However, the catheter

shaft (22) of the catheter member (12) typically includes more than one lumen, such as dual lumen or triple-lumen permitting bi-directional fluid flow. During an exemplary hemodialysis procedure, a multiple lumen catheter is inserted into a patient and blood is withdrawn through an arterial lumen of the catheter. The removed blood is directed to a hemodialysis unit which dialyzes, or purifies, the blood to remove waste, and toxins. The dialyzed blood is returned to the patient through a venous lumen of the catheter. In some embodiments, the catheter shaft (22) defines three independent lumens including two large lumens and one small lumen. At the catheter hub (10), each lumen (24) is connected to a separate extension line (14).

The catheter shaft (22) is formed of a flexible material such that the catheter shaft (22) is bendable into a “U” shape or an “L” shape, as will be further described. Suitable materials include, but are not limited to, polyurethanes including polyether polyurethanes, polyester polyurethanes, polyurethaneureas; polyvinylpyrrolidones; polyvinyl alcohols; polyethylene glycols; polypropylene glycols; polyoxyethylenes; polyacrylic acid; polyacrylamide; polytetrafluoroethylene; polyvinyl chloride (PVC); polyvinylacetate; poly(ethylene terephthalate); silicone; polyesters; polyamides; polyureas; styrene-block copolymers; polymethyl methacrylate; acrylic-butadiene-styrene copolymers; polyethylene; polystyrene; polypropylene; natural and synthetic rubbers such as latex rubbers; acrylonitrile rubber; and copolymers and mixtures of any of the above.

As shown in FIG. 5, the catheter (2) is partially insertable into an outer tubing (3). The outer tubing (3) is configured to receive and accommodate the catheter (2) therethrough in a substantially concentric relationship relative to each other. In some embodiments, the outer tubing (3) comprises a first tubing end (26), a second tubing end (28), and an elongated tubing body (30) extending between the first and second tubing ends (26, 28). The tubing body (30) defines a tubing bore (32) extending throughout its length to receive and accommodate the catheter shaft (22) therethrough.

In some embodiments, the outer tubing (3) is formed of a flexible material such that the outer tubing (3) is bendable into a “U” shape or an “L” shape, as will be further described. Suitable materials include, but are not limited to, polyurethanes including polyether polyurethanes, polyester polyurethanes, polyurethaneureas; polyvinylpyrrolidones; polyvinyl alcohols; polyethylene glycols; polypropylene glycols; polyoxyethylenes; polyacrylic acid; polyacrylamide; polytetrafluoroethylene; polyvinyl chloride (PVC); polyvinylacetate; poly(ethylene terephthalate); silicone; polyesters; polyamides; polyureas; styrene-block copolymers; polymethyl methacrylate; acrylic-butadiene-styrene copolymers; polyethylene; polystyrene; polypropylene; natural and synthetic rubbers such as latex rubbers; acrylonitrile rubber; and copolymers and mixtures of any of the above.

In some embodiments, the outer tubing (3) comprises first and second tubing caps (34, 36) removably attachable at the first and second tubing ends (26, 28). When attached, the first and second tubing caps (34, 36) seal the catheter shaft (22) within the tubing bore (32) of the outer tubing (3). The outer tubing (3) serves as a reservoir to store fluid. Suitable fluids include, but are not limited to, medium, saline, infusate, human plasma, human serum, artificial urine, artificial saliva, inoculum, and the like.

In some embodiments, the first tubing cap (34) comprises an upwardly extending arm (38) and connector (40) for removably attaching a first air vent (42) (FIG. 9). The first air vent (42) maintains the internal pressure of the outer tubing (3) in balance with the ambient pressure to avoid the deformation of the outer tubing (3), and facilitates draining and refilling of the outer tubing (3). The first air vent (42) comprises an elongated tube (44) which extends upwardly above the fluid within the outer tubing (3) to prevent contamination from the fluid during handling, thereby maintaining sterility of the first air vent (42). The first air vent (42) is sealed by a first vent cap (46).

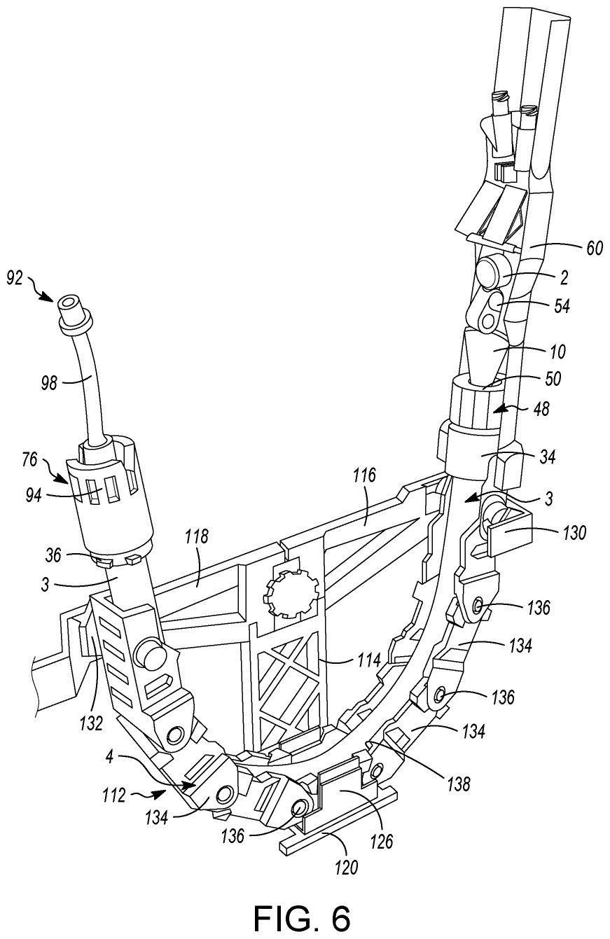

In some embodiments, a first end cap (48) is configured to be removably attachable to the first tubing cap (34) (FIGS. 6, 11, and 14). In some embodiments, the first end cap (48) at the first tubing end (26) defines a port (50) configured for allowing the catheter shaft (22) to pass therethrough. The port (50) is dimensioned to avoid any gap between the outer surface (52) of the catheter shaft (22) and the first end cap (48), and to position or isolate the catheter hub (10), extension lines (14), luer hubs (16), and associated clips (54) outside the outer tubing (3).

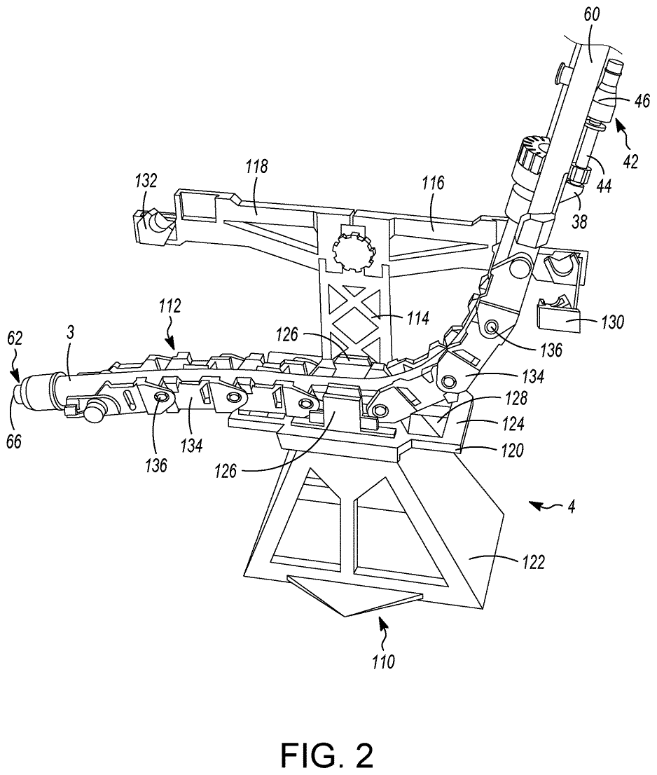

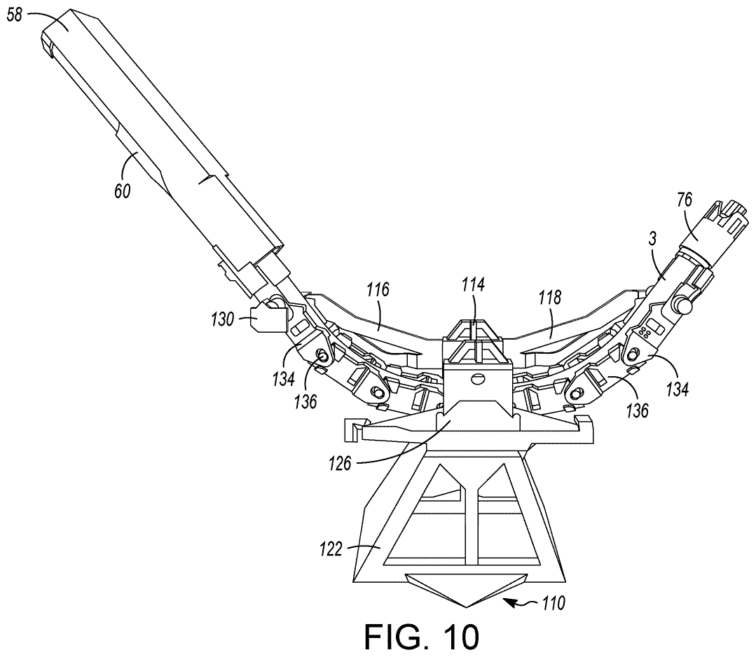

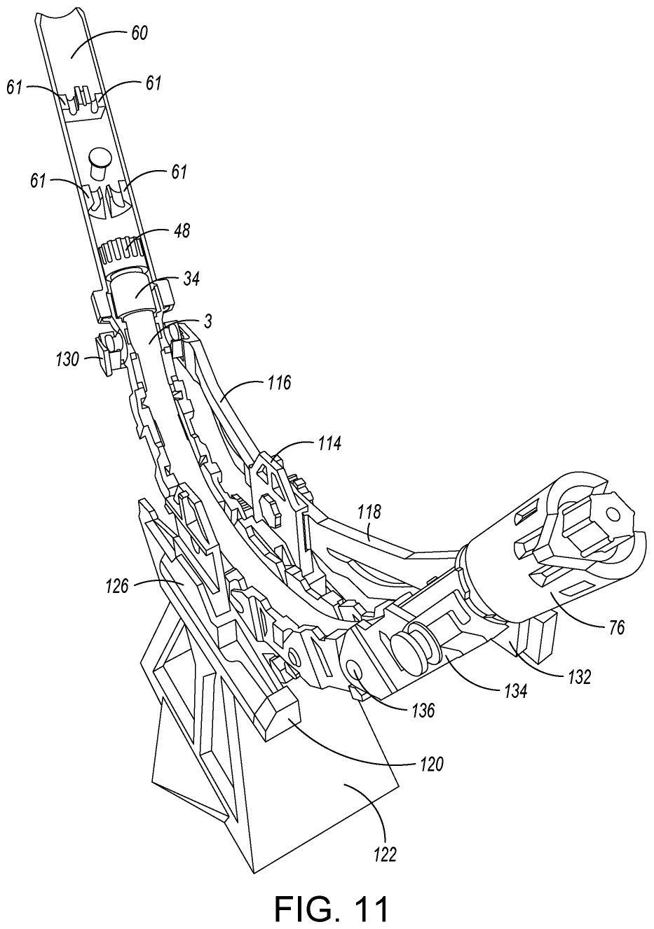

In some embodiments, a cover (56) is configured to accommodate catheter components including the first tubing cap (34), first end cap (48), catheter hub (10), extension lines (14), luer hubs (16), and clips (54) (FIGS. 1, 6, 10, 12, and 13). Such components are typically kept outside the patient's body when the catheter (2) is used during a medical procedure. The cover (56) comprises a front portion (58) and a back portion (60) which are removably attachable (FIGS. 1, 10, 12, and 13). FIGS. 2-4, 6, 9, 11, and 14 illustrate the cover (56) with the front portion (58) removed to show the positioning of the various catheter components. The back portion (60) may include one or more clips (61) (FIG. 11) to support a specific type of catheter (2) (e.g., dual lumen or triple-lumen). When the front and back portions (58, 60) are attached, the cover (56) encases the components to maintain their sterility during testing (FIGS. 1 and 10). The upwardly extending arm (38) and first air vent (42) are positioned behind the back portion (60) of the cover (56) (FIGS. 1-3 and 9). In some embodiments, the cover (56) is formed of a substantially rigid material to support and stabilize the various catheter components in a vertical direction during testing. Suitable materials include, but are not limited to, polyethylene terephthalate glycol, polylactic acid, thermoplastic polyurethane, and the like.

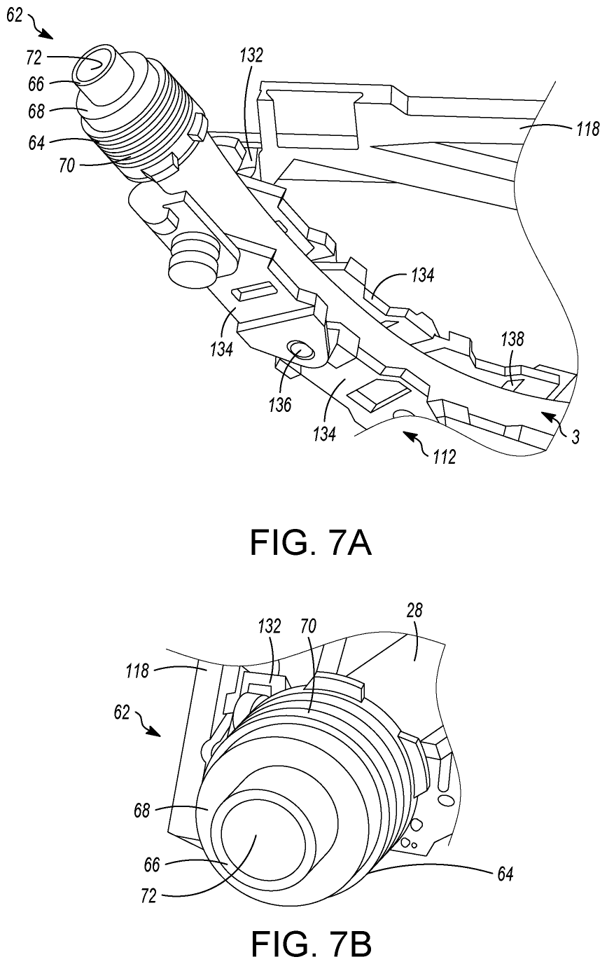

In some embodiments, the second tubing end (28) comprises a spout (62) (FIGS. 7A-B and 12). In some embodiments, the spout (62) comprises a substantially cylindrically-shaped housing (64) defining a central outlet (66), a shoulder (68), external threads (70), and a conduit (72) extending therethrough for allowing the drainage of fluid from the outer tubing (3). The central outlet (66) projects upwardly from the shoulder (68) and has a diameter which is narrower than the diameter of the shoulder (68). This narrower diameter of the central outlet (66) facilitates pouring of fluid from the outer tubing (3) without spillage or leakage of the fluid, thereby preventing potential contamination of the external threads (70) with the fluid. The external threads (70) mate with corresponding internal threads (not shown) of a second end cap (76).

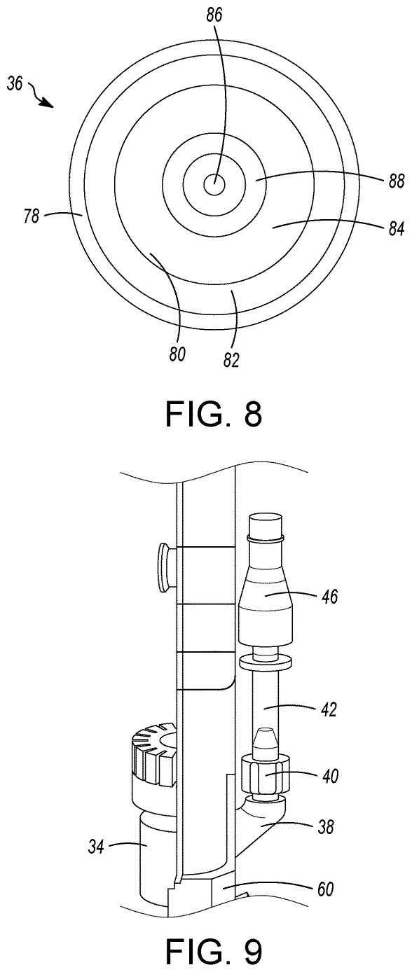

In some embodiments, the second tubing cap (36) is configured for receiving and accommodating the spout (62). The second tubing cap (36) comprises a substantially cylindrically-shaped housing (78) defining a cavity (80), internal threads (82), an internal seat (84), and a central aperture (86) (FIG. 8). The cavity (80) is sized to receive the spout (62) therein. A gasket (88) fits within the cavity (80). When the second tubing cap (36) is attached to the spout (62), the central outlet (66) of the spout (62) abuts the gasket (88) which seals against the spout (62), thereby preventing leakage of fluid from the outer tubing (3) during testing. The second tubing cap (36) defines an internal seat (84) which rests against the shoulder (68) of the spout (62). The internal threads (82) of the second tubing cap (36) are dimensioned to engage the corresponding external threads (70) of the spout (62). The second tubing cap (36) is attached to the spout (62) by engaging the internal threads (82) of the second tubing cap (36) with the external threads (70) of the spout (62). The central aperture (86) allows the passage of air from the outer tubing (3) through a second air vent (92).

In some embodiments, a second end cap (76) is configured to be removably attachable to the second tubing cap (36) (FIGS. 6, 10, and 11). The second end cap (76) comprises upwardly projecting walls (94) defining internal threads (not shown) for engaging corresponding external threads (not shown) of the second air vent (92). The second air vent (92) maintains the internal pressure of the outer tubing (3) in balance with the ambient pressure to avoid the deformation of the outer tubing (3), and facilitates draining and refilling of the outer tubing (3). The second air vent (92) comprises an elongated tube (98) which extends upwardly above the fluid to prevent contamination from the fluid during handling, thereby maintaining sterility of the second air vent (92). The second air vent (92) is sealed by a second vent cap (100).

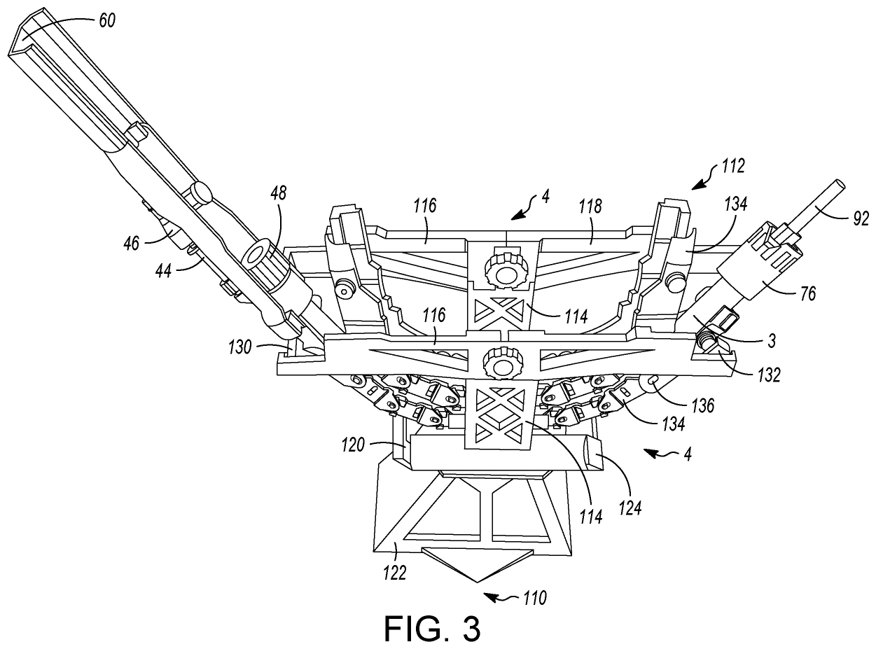

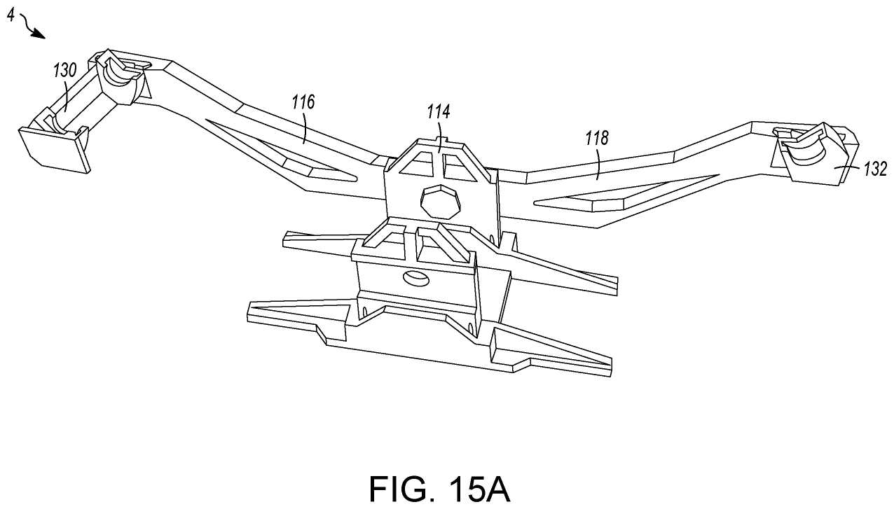

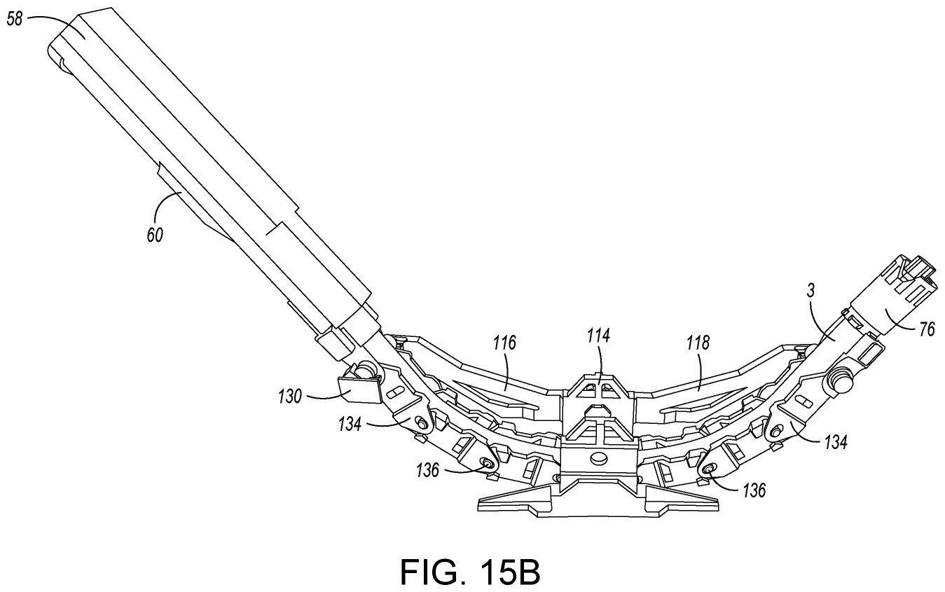

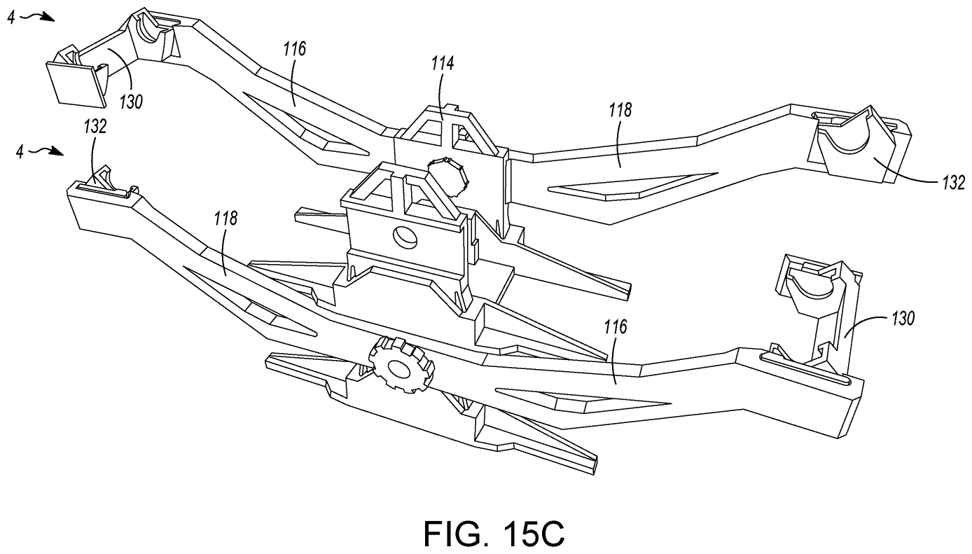

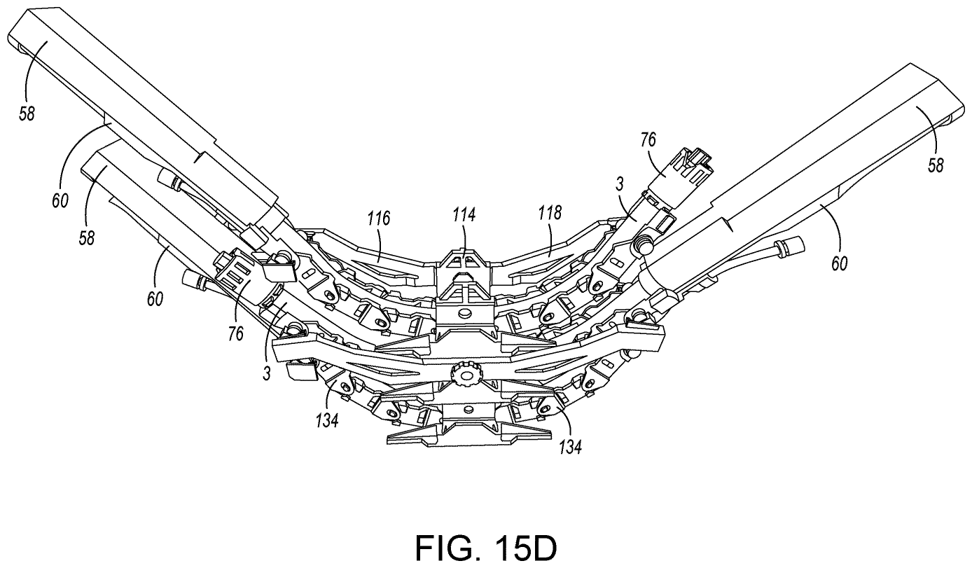

The assembled outer tubing (3), catheter (2), and various components described above are removably secured within a stand (4) (FIGS. 1-3, 6, and 10-15D). In some embodiments, the stand (4) is configured to stay horizontal or level on a workspace of a biosafety cabinet (5) when in use. The stand (4) is also designed to be stackable with other stands (4) to take up less space when placed in an incubator (FIGS. 3 and 15C-D). The dimensions of the stand (4) may be increased or decreased as may be required to satisfy any particular design objectives.

In some embodiments, the stand (4) generally comprises a base (110), a holder (112), a frame (114), and first and second clamp arms (116, 118). In some embodiments, the base (110) comprises a top portion (120) and a bottom portion (122) (FIGS. 2, 3, and 10-14). In some embodiments, the bottom portion (122) is configured to confer stability when in use. In some embodiments, the bottom portion (122) is configured in the form of a quadrilateral or four-sided polygon having four edges or sides and four corners or vertices, when viewed in cross-section. While FIGS. 2, 3, and 10-14 illustrate a quadrilateral bottom portion (122), it will be appreciated by those skilled in the art that other shapes or configurations (FIGS. 1 and 6) for the bottom portion (122) are included within the scope of the invention.

In some embodiments, the top portion (120) is substantially rectangular-shaped. While the Figures illustrate a rectangular-shaped top portion (120), it will be appreciated by those skilled in the art that other shapes for the top portion (120) are included within the scope of the invention. In some embodiments, the top portion (120) comprises a flat, horizontal upper surface (124) for supporting multiple components thereon. In some embodiments, the upper surface (124) supports a pair of opposed clips (126) positioned on the perimeter of the upper surface (124) for detachably retaining the holder (112). In some embodiments, the top portion (120) defines a window (128) for providing space and accommodating the holder (112) as the holder (112) is manipulated from a “U”-shaped position to an “L”-shaped position, or vice versa.

In some embodiments, the upper surface (124) supports the frame (114) which is positioned on the perimeter of the upper surface (124) for mounting the first and second clamp arms (116, 118). In some embodiments, the frame (114) comprises a diamond mesh pattern to keep the apparatus (1) lightweight, although other patterns are included within the scope of the invention. The frame (114) supports the outwardly extending first and second clamp arms (116, 118) mounted thereon by suitable attachment means including, but not limited to, a bolt. Each of the first and second clamp arms (116, 118) comprises first and second clamping and releasing means (130, 132) which are configured to clamp the first and second tubing ends (26, 28) vertically to retain fluid, and to release the first and second tubing ends (26, 28) from the vertical to a horizontal or lowered position to allow for fluid to drain. In some embodiments, the first and second clamping and releasing means (130, 132) comprise clip clamps.

In some embodiments, the holder (112) is configured to be reticulating. As used herein, the term “reticulating” means resembling a net arrangement or pattern. In some embodiments, the holder (112) comprises a plurality of links (134) which are coupled by movable joints (136) and define a channel (138) for receiving the outer tubing (3) and catheter (2) therein. The movable joints (136) enable manipulation of the holder (112) from a first “U” shaped position to a second “L-shaped” position, or vice versa. In the “U” shaped position, the first and second tubing ends (26, 28) of the outer tubing (3) are held vertically by the first and second clamping and releasing means (130, 132) to prevent release of fluid from within the outer tubing (3) (FIG. 1). In the “L” shaped position, the second tubing end (28) of the outer tubing (3) is released from the second clamping and releasing means (130) and lowered to allow draining of fluid through the spout (62) from within the outer tubing (3) (FIG. 2). Concomitantly, the flexible outer tubing (3) and catheter shaft (22—FIG. 4) held within the channel (138—FIG. 6) of the holder (112) assume a corresponding “U” shape in the first position of the holder (112), and a corresponding “L” shape in the second position of the holder (112).

Embodiments of the present invention are described in the following Examples, which are set forth to aid in the understanding of the invention, and should not be construed to limit in any way the scope of the invention as defined in the claims which follow thereafter. The Examples describe an exemplary apparatus, system, and method for in vitro testing of the internal and external surfaces of catheters simultaneously to evaluate reduction in microbial colonization. Notably, the apparatus, system, and method are conducted using minimal set volumes of fluid, thereby obviating the need for a continuous flow of fluid and the associated expense, time, and waste. The apparatus has been specially designed to operate with minimal set volumes of fluid, to be sealed to prevent or reduce the risk of contamination and spillage, and to be incubated with or without agitation for predetermined time periods before draining and replacing the fluid.

### Example 1

### i) Preparation of Outer Tubing and Catheter

Silicone tubing was cut to a desired appropriate length and sterilized in an autoclave for use as the “outer tubing.” An acute hemodialysis catheter (“AHDC”) including a shaft defining a triple lumen (e.g., two large lumens and one small lumen) was used for testing. The proximal end of the AHDC was secured to a first end cap, with the catheter shaft being positioned within the silicone tubing and the catheter hub, extension lines, and luer hubs positioned outside the silicone tubing (FIGS. 4 and 5). The silicone tubing supporting the AHDC (FIG. 5) was then mounted into the holder of the testing apparatus. The appropriate volume of conditioning fluid or microbial inoculum was sufficient to fill the silicone tubing such that the external surface of the catheter shaft was surrounded by the conditioning fluid or microbial inoculum for the desired appropriate times.

### (ii) Pre-Treatment and Simulated Dialysis

The air vent in the proximal cap was opened. Needleless connectors (NLCs) were attached to all three luer hubs of the AHDC. An appropriate volume of 0.9% saline was flushed through each lumen of the AHDC, allowing the excess saline to drain out of the AHDC. An appropriate volume of simulated dialysis fluid was flushed through each lumen of the AHDC, allowing the excess fluid to drain out of the AHDC. The residual fluid remained trapped in the AHDC due to the NLCs. An appropriate volume of simulated dialysis fluid was added to the silicone tubing to surround the external surface of the AHDC. A cap was placed on the distal end of the silicone tubing. The air vent in the proximal cap was then closed. The silicone tubing supporting the AHDC was mounted into the holder of the testing apparatus, ensuring that the entire length of the AHDC was covered by the conditioning fluid. The testing apparatus was placed on a rotary shaker set to 50±10 rpm.

After 3±0.5 hours, the testing apparatus was transferred to a biosafety cabinet. An appropriate volume of simulated dialysis fluid was withdrawn from each of the lumens and discarded. An appropriate volume of saline was flushed through each of the lumens. The residual saline remained trapped in the AHDC due to the NLCs. The silicone tubing supporting the AHDC was mounted into the holder of the testing apparatus. The testing apparatus was then placed on a rotary shaker set to 50±10 rpm and incubated at 37±2° C. for 24±4 hours.

(iii) Preconditioning Fluid Changes

Following incubation, the testing apparatus was transferred to the biosafety cabinet. The extraluminal conditioning fluid was drained by removing a cap on the distal end and allowing the fluid to drain out. An appropriate volume of saline was flushed through each lumen of the AHDC, allowing the excess fluid to drain out. The residual saline remained trapped in the AHDC due to the NLCs. The extraluminal conditioning fluid was replaced by adding an appropriate volume of simulated dialysis fluid to the silicone tubing. The silicone tubing supporting the AHDC was mounted into the holder of the testing apparatus. The testing apparatus was then placed on a rotary shaker set to 50±10 rpm and incubated at 37±2° C. for 24±4 hours.

### (iv) Simulated Dialysis and Preconditioning Changes

Following incubation, the testing apparatus was transferred to the biosafety cabinet. An appropriate volume of saline was withdrawn from each lumen and discarded. An appropriate volume of simulated dialysis fluid was flushed into each of the lumens. These volumes were intended to fill the lumens, without any excess. The silicone tubing supporting the AHDC was mounted into the holder of the testing apparatus. The testing apparatus was then placed on a rotary shaker set to 50±10 rpm and incubated at 37±2° C. for 3±0.5 hours.

Following incubation, the testing apparatus was transferred to the biosafety cabinet. The extraluminal conditioning fluid was drained by removing the cap on the distal end and allowing the fluid to drain out. An appropriate volume of air was flushed through the lumens followed by an appropriate volume of saline, allowing the excess fluid to drain out of the AHDC. The residual saline remained trapped in the AHDC due to the NLCs. The extraluminal conditioning fluid was replaced by adding an appropriate volume of simulated dialysis fluid to the silicone tubing. The silicone tubing supporting the AHDC was mounted into the holder of the testing apparatus. The testing apparatus was then placed on a rotary shaker set to 50±10 rpm and incubated at 37±2° C. for 72±4 hours or 24±4 hours.

### (v) Subculture

Using a cryogenic stock (at approximately −80° C.), first subcultures of microbial organisms were streaked out on appropriate media in plates. The microbial organisms may include, but are not limited to, Staphylococcus aureus, Acinetobacter baumannii, Enterococcus faecalis, Escherichia coli, Staphylococcus epidermidis, Klebsiella pneumoniae, Pseudomonas aeruginosa, Candida auris, Candida albicans, Aspergillus niger, and Fusarium oxysporum. The plates were incubated at appropriate times and temperatures, wrapped in Parafilm™, and stored at about 4° C. From each first subculture, a second subculture was streaked out on appropriate media in plates. The plates were incubated at appropriate times and temperatures. Each second subculture was used within 32 hours starting from the time it was first removed from incubation.

### (vi) Inoculum Preparation

From each second subculture, the study organisms were inoculated into appropriate sterile growth media for the selected microorganisms. All organisms were incubated at 37±2° C. on a rotary shaker set at approximately 110 rpm for 20±2 hours to yield an intended inoculum density. Each inoculum was adjusted to a chosen cell density appropriate to the clinical application by diluting the organisms. The cell density was confirmed by standard counting techniques.

(vii) Rinse and Challenge

Following the desired conditioning time, the extraluminal conditioning fluid was drained out of the outer tubing by removing the cap on the distal end and allowing the fluid to drain out. An appropriate volume of saline was flushed followed by an appropriate volume of air through each lumen of the AHDC, allowing the excess fluid to drain out. The external surface of the AHDC was rinsed for 30-60 seconds by filling the same outer tubing used for preconditioning with an appropriate volume of sterile saline.

After rinsing, the AHDC was transferred to a second sterile silicone outer tubing and a cap was applied on the distal end of the second tubing(The NLCs were removed from the luer hubs. An appropriate volume of inoculum was flushed through each of the lumens of the AHDC, retaining the excess fluid within the AHDC. After each flush, the extension tube was clamped to ensure that the inoculum remained in the AHDC, the syringe was removed, a cap was placed on the luer hub, and the clamp on the extension tube was disengaged.

An appropriate volume of the challenge inoculum was added to the outer tubing. This resulted in the distal tip of the catheter shaft of the ADHC being fully exposed to the inoculum, but a small portion of the proximal shaft was not covered by the inoculum. This was to ensure that the inoculum did not contact the cap during the challenge. The silicone tubing supporting the AHDC was mounted into the holder of the testing apparatus. The testing apparatus was then placed on a rotary shaker set to 50±10 rpm and incubated at 37±2° C. for 24±2 hours.

(viii) Recovery

Following incubation, planktonic recovery was conducted. The challenge medium was collected by lowering the distal end of the outer silicone tubing and draining the challenge medium into a sterile planktonic recovery collection tube. An appropriate volume of saline was flushed followed by an appropriate volume of air through each lumen of the AHDC. The excess fluid was collected in the same collection tube containing the extraluminal challenge medium.

In triplicate, a sample from each planktonic recovery collection tube was mixed with 2× neutralizer. A serial dilution was prepared and spot or spread plated on appropriate agar. The plates were incubated at appropriate times and temperatures and then counted. The data were evaluated as total CFU recovered/apparatus. Log₁₀ reduction was compared to control simple counts.

### Example 2

The steps in Example 1 can be conducted using minimal set volumes of fluid, thereby obviating the need for a continuous flow of fluid and the associated expense, time, and waste, as required by conventional methods. The dimensions of the apparatus described herein may be increased or decreased as may be required to satisfy any design objectives. The volumes of fluid may be dictated by the dimensions of the silicone tubing and AHDC. Example 2 sets out exemplary minimal set volumes of fluid used with silicone tubing and AHDC having specified dimensions.

In step (i), a silicone tubing having an inner diameter of 10 mm and length of 35 cm was used as the outer tubing. An AHDC having a length of 30 cm was tested. 20 mL of conditioning fluid or microbial inoculum was sufficient to fill the silicone tubing.

In step (ii), 5 mL of each of 0.9% saline and simulated dialysis fluid was flushed through the AHDC. 20 mL of simulated dialysis fluid was added to the silicone tubing to surround the external surface of the AHDC. Following the agitation step, about 1.0 to 4.0 mL of simulated dialysis fluid was withdrawn from the lumens and discarded. About 1.0 to 3.0 mL of saline was flushed through the lumens.

In step (iii), 5 mL of saline was flushed through the AHDC. The extraluminal conditioning fluid was replaced by adding 20 mL of simulated dialysis fluid to the silicone tubing.

In step (iv) following incubation, about 1.0 to 4.0 mL of saline was withdrawn from the lumens and discarded. About 1.0 to 3.0 mL of simulated dialysis fluid filled the lumens. Following incubation, at least 5.0 mL of air was flushed through the lumens followed by 5.0 mL of saline. The extraluminal conditioning fluid was replaced by adding 20 mL of simulated dialysis fluid to the silicone tubing.

In step (vii), 5.0 mL of saline was flushed followed by at least 5.0 mL of air through the lumens of the AHDC. The external surface of the AHDC was rinsed with 20 mL of sterile saline. After rinsing, the AHDC was transferred to a second sterile silicone outer tubing. 5.0 mL of inoculum was flushed through each of the lumens of the AHDC. 11.0 mL of the challenge inoculum was added to the extraluminal portion of the outer tubing.

In step (viii), 5.0 mL of saline was flushed followed by at least 5.0 mL of air through each lumen of the AHDC.

It should be apparent, however, to those skilled in the art that many more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or integrated with other elements, components, or steps that are not expressly referenced. Accordingly, various changes and modifications can be made to the exemplary embodiments and uses without departing from the scope of the invention as defined in the claims that follow.

What is claimed is:

1. An apparatus for testing a catheter comprising:
an outer tubing configured for receiving the catheter; and
a stand comprising a reticulating holder configured for receiving the outer tubing and the catheter therein, and clamping and releasing means for (i) securing the holder, the outer tubing, and the catheter into a first “U” shaped position for conditioning and/or challenging internal and external surfaces of the catheter simultaneously, and (ii) releasing the holder, the outer tubing, and the catheter into a second “L” shaped position for draining fluid.

2. The apparatus of claim 1, wherein the reticulating holder comprises a plurality of links coupled by a plurality of movable joints and defining a channel for receiving the outer tubing and the catheter therein.

3. The apparatus of claim 1, wherein the catheter comprises a catheter hub, an elongated catheter member extending distally from the catheter hub, one or more extension lines, and one or more luer hubs.

4. The apparatus of claim 1, wherein the outer tubing comprises a first tubing end, a second tubing end, and an elongated tubing body extending between the first and second tubing ends and defining a tubing bore for receiving and accommodating the catheter therethrough.

5. The apparatus of claim 4, wherein the outer tubing further comprises first and second tubing caps removably attachable at the first and second tubing ends for sealing the catheter in the tubing bore.

6. The apparatus of claim 5, wherein the first tubing cap comprises an upwardly extending arm for removably attaching a first sealable air vent extending upwardly above the fluid in the outer tubing.

7. The apparatus of claim 5, further comprising a first end cap removably attachable to the first tubing cap, the first end cap defining a port for allowing a catheter member to pass therethrough, and isolating a catheter hub, extension lines, and luer hubs externally from the outer tubing.

8. The apparatus of claim 7, further comprising a cover for encasing the first tubing cap, the first end cap, the catheter hub, the extension lines, and the luer hubs.

9. The apparatus of claim 5, wherein the second tubing end comprises a spout including a cylindrically-shaped housing defining a central outlet, a shoulder, external threads, and a conduit extending therethrough for allowing the drainage of the fluid from the outer tubing.

10. The apparatus of claim 9, wherein the central outlet projects upwardly from the shoulder and has a diameter narrower than the diameter of the shoulder.

11. The apparatus of claim 9, wherein the external threads of the spout mate with corresponding internal threads of a second end cap.

12. The apparatus of claim 9, wherein the second tubing cap comprises a cylindrically-shaped housing defining a cavity for receiving the spout, internal threads for engaging the external threads of the spout, an internal seat for resting against the shoulder of the spout, a central aperture allowing passage of air from the outer tubing through a second sealable air vent, and a gasket for sealing the spout against the second tubing cap.

13. The apparatus of claim 12, wherein the second end cap is removably attachable to the second tubing cap, and comprises upwardly projecting walls defining internal threads for engaging external threads of the second sealable air vent extending upwardly above the fluid within the outer tubing.

14. The apparatus of claim 1, wherein the stand further comprises a base including a top portion and a bottom portion, wherein the top portion comprises an upper surface for supporting a pair of opposed clips for detachably retaining the reticulating holder.

15. The apparatus of claim 1, wherein the stand further comprises a frame for mounting first and second clamp arms including first and second clamping and releasing means for clamping and releasing the reticulating holder, the outer tubing, and the catheter.

16. A method of testing a catheter comprising:
providing the catheter and an apparatus for testing a catheter wherein the apparatus comprises an outer tubing configured for receiving the catheter; and a stand comprising a reticulating holder configured for receiving the outer tubing and the catheter therein, and clamping and releasing means for (i) securing the holder, the outer tubing, and the catheter into a first “U” shaped position for conditioning and/or challenging internal and external surfaces of the catheter simultaneously, and (ii) releasing the holder, the outer tubing, and the catheter into a second “L” shaped position for draining fluid;
optionally, conditioning lumens of the catheter shaft and an extraluminal portion of the catheter shaft within the outer tubing with one or more conditioning fluids;
challenging with a medium comprising a microbial inoculum by filling the lumens of the catheter shaft and the outer tubing with the medium to contact the internal and external surfaces of the catheter shaft simultaneously;
incubating the apparatus for a predetermined time sufficient for microbial colonization on the internal and external surfaces of the catheter shaft;
collecting the medium from the lumens of the catheter shaft and the outer tubing, the medium comprising planktonic microorganisms collected in an initial recovery step, or microorganisms dislodged from the internal and external surfaces of the catheter shaft collected in a subsequent recovery step; and
quantifying microbial colonization from the collected medium.

17. The method of claim 16, wherein the catheter is selected from a hemodialysis catheter comprising an acute hemodialysis catheter, a central venous catheter, a peripherally inserted central catheter, a peripheral intravenous catheter, an endotracheal tube, or a Foley catheter.

18. The method of claim 16, wherein conditioning comprises pre-treating and incubating the lumens of the catheter shaft and the extraluminal portion of the catheter shaft within the outer tubing with the one or more conditioning fluids for a predetermined time.

19. The method of claim 18, wherein the one or more conditioning fluids are selected from saline, infusate, human plasma, human serum, artificial urine, or artificial saliva.

20. The method of claim 18, further comprising draining the one or more conditioning fluids from the lumens of the catheter shaft and the outer tubing before challenging with the medium comprising the microbial inoculum.
